(19) United States
(12) Patent Application Publication
(10) Pub. No.: US 2006/0166204 A1
(43) Pub. Date: Jul. 27, 2006

(54) Parkinson's disease susceptibility haptotype as a tool for genetic screening

(75) Inventors: Hermona Soreq, Jerusalem (IL); Liat Ben-Moyal, Omer (IL); Boris Bryk, Jerusalem (IL); Alon Friedman, Gedera (IL)

Correspondence Address: COOPER & DUNHAM, LLP, 1185 AVENUE OF THE AMERICAS, NEW YORK, NY, 10036, US

(73) Assignees: Yissum Research Development Company of the Hebrew University of Jerusalem, Jerusalem (IL); Mor Research Applications Ltd., Petach Tikva (IL); Ben-Gurion University of Negev, Research & Development Authority, Beersheva (IL)

(21) Appl. No.: 10/529,511

(22) PCT Filed: Sep. 24, 2003
(86) PCT No.: PCT/IL03/00764

§ 371 Date: Oct. 24, 2005

(30) Foreign Application Priority Data
Sep. 26, 2002 (IL) 151955

(51) Int. Cl. C12Q 1/68

(52) U.S. Cl. 435/6

(57) ABSTRACT

Specific PON1 and ACHE alleles segregate in linkage, forming an haplotype which directly correlates with higher susceptibility to develop Parkinson's Disease (PD). This PD-susceptibility haplotype is herein presented as a tool for predicting the risk of developing Parkinson's Disease and its severity, both for an individual and for the population in general. Thus, the present invention provides the use of said PD-susceptibility haplotype in diagnostic and screening methods.

### FIELD OF THE INVENTION

The present invention relates to a genetic predisposition to Parkinson's Disease, involving a haplotype found on Chromosome 7.

### BACKGROUND OF THE INVENTION

All publications mentioned throughout this application are fully incorporated herein by reference, including all references cited therein.

Parkinson's Disease (PD) is a late-onset, progressive neurodegenerative disorder consisting of a variable combination of clinical symptoms: resting tremor, muscular rigidity, bradykinesia and a characteristic disturbance in gait and posture. The disease generally commences in the middle or late life and leads to progressive disability with time. It has an equal sex distribution, occurs in all ethnic groups, and has a prevalence of 1-2 per 1000 in the general population [Lang, A. E. and Lozano, A. M. (1998) N. Engl. J. Med. 339:1044-1053, Aminoff M. J. (2001) Parkinson's disease and other extrapyramidal disorders. In: Braunwald E. et al. (eds) Harrison's principles of internal medicine. McGraw Hill, pp. 2399-2406].

Pathologically, PD is characterized by the progressive death of selected populations of neurons, especially dopaminergic neurons of the pars compacta of the substantia nigra. Other regions, such as the aminergic brain-stem nuclei (both catecholaminergic and serotoninergic), the cholinergic nucleus basalis of Meynert, hypothalamic neurons, and small cortical neurons particularly in the cingulate gyrus and entorhinal cortex), as well as the olfactory bulb, sympathetic ganglia, and parasympathetic neurons in the gut, may also suffer neuronal loss. Neuronal degeneration within the pars compacta of the substantia nigra leads to a reduction in dopamine levels within the striatum, and especially the Putamen, thereby accounting for the typical akinesia and rigidity seen in the disease. Other pathological features include the appearance of Lewy neuritis as well as eosinophllic hyaline inclusions called Lewy bodies.

Neuronal death may be caused by a variety of possible mechanisms, for instance mitochondrial dysfunction [Vingerhoets F. J. G., et al. (1994) Ann. Neurol. 36:765-70], the metabolism of oxidants produced in the course of neural metabolism [Jenner P. and Olanow C. W. (1996) Neurology, 47(Suppl 3): S161-S170], possible deficiencies in neurotrophic factors, resulting in decreased neural repair and leading to degradation of dopaminergic cells [Aminoff (2001) id ibid.], and immune factors found where there is progressing neuronal loss, as seen through an increase in cytokines such as interleukin-1 and tumor necrosis factor α in the pars compacta of the substantia nigra in parkinsonian patients [Lang A. E. and Lozano A. M. (1998) id ibid.].

In addition, various environmental factors are linked to the occurrence of Parkinson's disease. Living in rural regions was found to be associated with a higher risk of Parkinson's disease, as well as a possible connection with exposure to herbicides, pesticides and well water [Betarbet, R. et al. (2000) Nature Neurosci. 3:1301-6; Menegon, A. et al. (1998) Lancet 352:1344-6.]

Age appears to be the most significant risk factor for Parkinson's disease, with am increase in prevalence up to 1-2 per 100 in people over the age of 65. Curiously, smoking is an environmental factor with a negative risk, since it has been found that the odds ratio for ever having smoked in Parkinson's patients, as compared with general population, is 0.5 [Tanner C. M. et al. (1997) Neurology, 48(Suppl):A333; Le Couteur D. G. et al. (2002) Rev. Environ. Health 17:51-64].

Genetic Factors of Parkinson's Disease

Genetic factors have proven to have an important role in Parkinson's disease.

Studies conducted with monozygotic twins showed a higher incidence of Parkinson's disease in one twin with young onset disease [Tanner C. M. (1997) id ibid.]. In Iceland, a study was conducted in 772 Parkinson's patients as well as 1000 independently drawn, matched control subjects. [Sveinbjornsdottir S. et al. (2000) N. Engl. J. Med. 343:1765-1770]. For each patient, the genealogical proximity from another patient of the same family was evaluated and a kinship coefficient obtained, demonstrating the genetic relationship between two patients, or the probability that a randomly selected allele from each member of a pair of subjects was from a common ancestor. The results showed that for the 772 Parkinson's patients, the average kinship coefficient was 2.7 (when multiplied by 10,000) as compared with 2.0±0.1 (mean±SD) in the control population (p value<0.001), thereby strengthening the notion that Parkinson's disease has a familial component.

Furthermore, it was shown that relatives of patients were at a higher risk of suffering from Parkinson's disease than the general population. This was most pronounced in siblings (p value<0.001) and first degree relatives (p value=0.001), than other relatives. Spouses showed no significant risk, thereby indicating the unlikelihood of the possibility that a shared environmental factor, late in life, accounts for Parkinson's disease, though the higher risk to siblings than to offspring indicates towards a possible environmental cause in early life [Tanner C. M. (1997) id ibid.; Jenner P. M. and Olanow C. W. (1996) Neurology 47:Suppl 3:S161-S170].

Several genes have been mapped that cause inherited monogenic forms of the disease. These genes are found to play a role in the formation of various proteins, such as α-synuclein and parkin, or to be linked to cellular processes as the electron transport chain in the mitochondria. Mutations in these different genes may cause varied probabilities of being afflicted by Parkinson's disease; as well as affecting its phenotype. However, these mutations are apparently responsible only for a small number of families. As of yet, the genetic basis for the sporadic form of the disease remains unknown.

Mutations in the Gene for α-synuclein

The gene enocoding the α-synuclein protein is located on the long arm of human chromosome 4. The gene encodes a small (14 kDa) highly conserved protein, found abundantly in many brain regions. This protein appears to take part in synaptic development, function, and plasticity.

Two point mutations in the gene were found in families of Parkinson's disease patients. Determination of the exact mutations revealed, in one Italian family, a single base mutation in position 209 from guanine to adenine (G209A) which resulted in an alanine to threonine substitution at position 53 (Ala53Thr) [Polymeropoulos M. H. et al. (1997) Science, 276:2045-47]. The second case involved two Greek families with another base change which resulted in an alanine to proline substitution at position 30 (Ala30Pro) [Kruger R. et al. (1998) Nat. Genet. 18(2):106-8].

Structurally, the wild type, as well as these mutant forms, are found in an unfolded conformation under physiological conditions. When the protein goes into a partially folded intermediate, a nucleation-dependent mechanism occurs, resulting in the formation of fibrils.

However, comparing the amino acid sequence of the wild type and mutant proteins revealed that the mutant forms differed from the wild type in their probability to aggregate. Both mutations showed a decrease in the probability to form a α-helix in the N-terminal regions, and an increased propensity to form β-sheets than the wild type protein. These structural changes, although not affecting the monomeric protein structure, have a direct influence on the increased tendency of the mutant proteins to form aggregates and fibrils, leading to the formation of Lewy bodies typically found in the disease [Spillantini M. G. et al. (1997) Nature, 388:839-40], thereby providing support for a direct role of α-synuclein aggregation in the etiology of Parkinson's disease.

PARK 2—Autosomal Recessive Juvenile Parkinsonism (AR-JP)

A second form of inherited Parkinson's disease is linked to a genetic locus mapped to the long arm of chromosome 6, the parkin gene. Clinically, these patients show an early age of onset, levodopa responsiveness, diurnal fluctuations of symptoms (becoming worse later in the day), as well as early and severe motor fluctuations and dyskinesia, with no evidence of Lewy bodies within the brain tissue. A recent work attempted to identify various mutations in the parkin gene [Lucking C. B. (2000) N. Engl. J. Med. 342:1560-7). 73 families (152 patients with Parkinson's disease and 63 unaffected relatives) that met the following criteria were examined: symptoms of parkinsonism which appeared before the age of 45, symptoms reduced by at least 30% by levodopa, a mode of inheritance compatible with autosomal recessive transmission, absence of extensor plantar reflexes, ophtalmoplegia, early dementia, or early autonomic failure. The families were gathered from countries around the globe. In addition, 100 patients with isolated Parkinson's disease were selected according to the same criteria. The screening for mutations was performed using a semiquantitative PCR assay for the detection of rearrangement of parkin exons. 19 different homozygous and heterozygous exon rearrangements were found, as well as 16 different point mutations, indicating that among patients with the autosomal recessive juvenile parkinsonism (AR-JP) variant, mutations in the parkin gene are frequent and varied. Clinical symptoms were also gathered from these patients and compared to identify any clinical distinctions. The patients with parkin mutations presented different symptoms from the isolated Parkinson's patients. No differences were found between patients bearing different mutations.

Through the use of semiquantitative PCR, mutations within the parkin gene were identified. The majority of mutations (70%) was found in patients suffering from AR-JP, thus pointing towards a link between the different mutations in the parkin gene and the appearance of the AR-JP variant of Parkinson's disease [Kitada T. et al. (1998) Nature 392:606-8]. However, although these patients showed early onset, no unique clinical signs were found to distinguish these patients from patients suffering from other causes of Parkinson's disease.

Mitochondrial Electron Transport Chain and Parkinson's Disease

Loss of electron transport chain activity is noted in various tissues in Parkinson's patients [Parker W. D. and Swerdlow R. H. (1998) Am. J. Hum. Genet. 62:758-62]. This biochemical defect is found in many tissues, including: platelets, lymphocytes brain, muscle, and fibroblasts. Evidence using immunoblot studies has demonstrated that a disruption of NADH ubiquinone oxidoreductase (complex I) subunits may be the cause of loss of mitochondrial activity. Furthermore, toxins such as the N-methyl-4-phenyl-1,2,3,6-tetrahydropyridine (MPTP) derived toxic metabolite MPP⁺ [Vays I. et al. (1986) J. Neurochem. 46:1501-7], together with various neuroleptic medications (haloperidol, chloropromazine, thiothixene) (Burkhardt C. et al. (1993) Ann. Neurol. 33:512-7], which all produce Parkinson's symptoms, have been shown to inhibit complex I activity in animal models. Therefore, disruption of normal mitochondrial activity may play a role in the appearance of Parkinson's disease.

Once complex I activity is decreased, the entire electron transport chain function may be perturbed, resulting in several consequences, a decrease in adenosine triphosphate (ATP) synthesis, followed by an accumulation of reactive oxygen-species (ROS) and oxidative stress within the cell. These effects were examined in immortalized human cell lines, depleted of mitochondrial DNA (mtDNA) by culturing for 3-4 months with ethidium bromide. mtDNA was reintroduced to these cells from platelets of Parkinson's disease patients (creating PD hybrid cells), resulting in an increase in ROS production when compared with controls [Cassarino D. S. et al. (1997) Biochim. Biophys. Acta, 1362:77-86). These changes may have a role in initiating apoptotic cell death within the afflicted regions of the brain. Moreover, such cells showed an increased sensitivity to MPP⁺ when compared to control cells, leading to apoptotic cell death. Thus, a possible interaction between a genetic abnormality and an environment agent may cause the disease phenotype.

Interestingly, an epidemiological study showed that in families where Parkinson's disease was present in parents and children, the disease seemed to be transmitted by the maternal lineage [Wooten G. F. et al. (1997) Ann. Neurol. 41:265-8]. Children with a mother who was ill showed an earlier onset of the disease when compared with their parent (p<0.001), while patients with a father who had the disease showed no significant difference in the age of onset. The maternal transmission might be associated with a mitochondrial defect.

Paraoxonase 1 (PON1)

The PON1 gene maps to chromosome 7q21.34 and it is localized 5,000 Kb downstream from the ACHE gene (FIG. 1). The PON gene family includes three or more genes of unknown function.

The gene product, PON1, is an aryldialkylphosphatase also known as paraoxonase, which hydrolyzes soman, sarin, paraoxon, diazinon, and other organophosphate (OP) substrates. PON1 is a glycoprotein associated with a subset of HDL molecules, which is produced and secreted in the liver, and exists in many tissues, particularly liver, kidney, small intestine and serum. This enzyme detoxifies OPs by hydrolyzing them, and prevents lipodoxidation of LDL and RDL [Mackness B. et al. (1998) Gen. Pharmac. 31, 329-336]. PON1 serum levels may vary by up to 40-fold from one individual to another.

Two coding region and five promoter polymorphisms are known in the PON1 gene (FIG. 2) (Brophy V. H. et al. (2001a) Pharmacogenetics 11-77-84; Brophy et al. (2001b) Am. J. Hum. Genet. 68:1428-1436]. In the coding region, the two polymorphisms are point mutations that result in amino acid changes. One is at amino acid position 55, L55M (CTG into ATG), while the second is at amino acid position 192, Q192R (CAA into CGA). Interestingly, the 55L allele is linked to the 192R allele [Akhmedova S. N. et al. (2001) J. Neurol. Sci. 184:179].

The PON1 192R alloenzyme is more active with the OPs paraoxon, methylparaoxon, chlorothion EPN oxon and armin, while the 192Q variant affords the carrier better protection against the OPs diazoxon, sarin and soman. Both alloenzymes hydrolyze phenyl acetate, chlorpyirifos oxon and naphtylacetate-2 the following substrates equally well [Costa L. G. et al. (1999) Chem. Biol. Interact.:119-120, 429-438; Mackness, B. et al. (1998) Gen. Pharmacol. 31:329-336].

The polymorphism at position 192 affects mRNA and protein levels, but not biochemical properties. The 192M variant is associated with low serum levels, and thus affords lower protection to xenobiotics [Costa et al. (1999) ibid.; Kondo and Yamamoto (1998) supra].

PON1 Promoter Polymorphisms

108 C/T: This polymorphism occurs at a putative SP1 binding site. The C variant produces more enzyme than the T variant (Brophy et al. (2001a) ibid.; Brophy et al. (2001b) ibid.].

909 G/C: This variant has no major effect on gene expression, except when it is linked to other polymorphisms.

162 A/G: The A variant promotes higher transcriptional activities than the G variant. It is localized in a putative NF1 binding site.

PON1 status is defined as the combination of the genotype and the phenotype, the latter being affected by a high fat diet and exposure to xenobiotics—which reduce PON1 expression regardless of the genotype. Species with low PON1 activities display higher OP sensitivity [Costa L. G. et al. (1987) Species differences in serum paraoxonase correlate with sensitivity to paraoxon toxicity. In: Costa L. G. (eds.) Toxicology of pesticides: experimental, clinical and regulatory perspectives. Springer-Verlag, Heidelberg, pp. 263-266]. Accordingly, PON1-l-knockout mice are five- to ten-fold more sensitive to the anti-ChEs diazoxon and chlorpyrifos oxon than wildtype mice [Furlong C. E. et al. (1998) Neurotoxicology 19(4-5):645-50; Costa L. G. et al. (1999) id ibid.). PON1 192R carriers have a higher risk for coronary arterial disease (CAD) [Nassar B. A. et al. (2002) Clin. Biochem. 5:205-209].

The PON1 alleles show different distributions in specific ethnic groups. No study has yet been performed in the Israeli population. A study comparing Chinese/Japanese and Caucasian populations showed the following incidence of polymorphism (Table 1) [Wang and Liu (2000) supra]:

TABLE 1

| Chinese/Japanese | Caucasian | PON1 Polymorphism |
|---|---|---|
| — | 0.46 | −909G |
| — | 0.54 | −909C |
| 0.10 | 0.23 | −162A |
| 0.90 | 0.77 | −162G |
| 0.48 | 0.50 | −108C |
| 0.52 | 0.50 | −108T |
| 0.94 | 0.64 | 55L |
| 0.06 | 0.36 | 55M |
| 0.40 | 0.73 | 192Q |
| 0.60 | 0.27 | 192R |

Other population studies have demonstrated the following results (Table 2):

TABLE 2

| Connection between PD and PON1 | Ethnic group | Reference |
|---|---|---|
| No link | Caucasian | (Taylor et al., |
| | | 2000)^{b} |
| No link | Chinese | (Wang et al., 2000)^{c} |
| Higher PON1 192R incidence in PD | Japanese | (Kondo et al., |
| patients. R homozygotes show 1.6 | | 1998)^{d} |
| ratio of increased risk.^{a} | | |
| Higher PON1 55M incidence in PD | Russian | (Akhmedova et al., |
| patients, with 2.19 odds ratio. | | 2001)^{e} |
| Yet higher PON1 55M incidence in | | |
| PD patients, with 2.19 odds ratio. | | |
| Yet higher PON1 55M in PD patients | | |
| with <51% early onset. Odds | | |
| ratio, 5.15. | | |
| Yet higher risk for those homozygous | | |
| for the 192Q allele. | | |

^{a}The risk ratio is the frequency of PD among the population under study compared to the frequency in a control population.
^{b}[Taylor M. C. et al. (2000) J. Neural Transm. 107: 979-983]
^{c}[Wang J. and Liu Z. (2000) Mov Disord15: 1265-1267]
^{d}[Kondo I. and Yamamoto M. (1998) Brain Res 806: 271-273]
^{e}[Akhmedova S. N. et al. (2001) J. Neurol. Sci. 184: 179-182]

PON1 Polymorphisms and the Gulf War Syndrome

In a study of Gulf War veterans, the R allele appeared more frequently in those affected by Gulf War Syndrome (GWS) than in those unaffected [Haley R. W. et al. (1999) Toxicol. Appl. Pharmacol. 157:227-233]. PON1 192Q carriers displayed lower PON1 serum activities in sick veterans when compared to healthy veterans.

PON1 and Parkinson's Disease

PON1 acts in the blood stream as a hydrolyser of various toxins which escape hepatic detoxification. The two PON1 polymorphisms in the coding region, an arginine to glutamine exchange at position 192 (Arg192Gln), and a methionine to leucine at position 55 (Leu55Met), influence the ability of PON1 to hydrolyze toxins, and may intensify the effects of pollutants, organophosphates and other environmental chemicals in the development of Parkinson's disease.

In a study to determine the association between the Leu55Met polymorphism and Parkinson's disease [Akhmedova S. N. et al. (2001) id ibid.], 117 unrelated idiopathic Parkinson's disease patients were analyzed. When compared to the distribution of the polymorphism in the general population, it was found that the Parkinson's Disease patients presented a higher incidence of the Leu55Met allele (p<0.003). Therefore, it seems that there is an association between the presence of the Leu55Met polymorphism and an increased risk of Parkinson's disease. The change in the activity of the enzyme, due to the polymorphism, does not ensure that one develops the disease, but it may indicate a higher sensitivity towards various chemicals and toxins which might trigger the disorder.

To conclude, it can be inferred that Parkinson's disease is manifested in a variety of forms, varying from the early onset disease with no presence of Lewy bodies, to the “classic” late onset manifestation. The occurrence of the disorder is influenced by many factors, including environmental, such as exposure to various chemicals (MPTP, neuroleptic drugs, organophosphates, and such), rural living (which might also be connected to an increase in exposure to such compounds) or occupational, as well as genetic predisposition. At least three genes induce increased risk for PD, while exposure to xenobiotics acts as a direct cause of PD in sporadic cases [Kaufer D. and Soreq H. (1999) Curr. Opin. Neurol. 12:739-743]. The relationship between the two causes is yet unknown.

ACHE

Acetylcholinesterase—(AChE), a type B-carboxylesterase, hydrolyses and inactivates acetylcholine (ACh). Changes in the level and mode of AChE gene expression are revealing indicators of alteration in cholinergic neurotransmission. For example, both acute psychological stress and exposure to organophosphate and carbamate AChE inhibitors (anti-AChEs) were found to induce rapid, yet long-lasting transcriptional AChE activation that was accompanied by a splicing shift, from the major AChE-S variant to the rare AChE-R mRNA and protein [Kaufer et al. (1998) Nature 393:373-7; Soreq, H. and Seidman, S. (2001) Nature Reviews in Neuroscience 2, 294-302].

The upstream promoter of the ACHE gene includes two mutations, one of which confers overproduction and hypersensitivity to anti-ChEs [Shapira M. et al. (2000) Hum. Mol. Genet. 9:1273-1281] (FIG. 3). Carriers of this ACHE promoter deletion express higher blood cell AChE levels and higher AChE activity (twice normal) in immortalized lymphocytes [Shapira (2001) id ibid.]. Transgenic human AChE-over-expressing mice suffer hypersensititivity to both carbamate and OP inhibitors and survive for a shorter time after injection of a lethal dose of diisopropyfluorophosphonate (DFP) than mice of the parent strain. Unlike normal mice, they are unable to induce AChE-R over-production following exposure, which contributes to their hypersensitivity.

ACHE Haplotypes

The different polymorphisms in the ACHE locus together form a haplotype with internal linkage that confers a common heritage. H332N (Asp for His) is the serological marker of the Yt^{b} blood group, while P446 is a silent mutation (FIG. 4b). These two mutations were reported to be 100% linked in the US population [Bartels et al. (1993) id ibid.]. In a later study, 80% linkage was shown for the promoter deletion with H332N [Shapira et al. (2001) id ibid.], linking these 3 sites.

ACHE and Parkinson's Disease

Exposure, even sub-acute, to xenobiotics may induce a toxic response of the intestine, the immune system, muscle or brain, and alter metabolic activities to the extent that longevity is affected, all depending on the genotype of the patient, type of exposure, and level of the toxin's penetrance.

Some of these xenobiotics are anti-cholinesterases, like, carbamates and organophosphates (OPs), which block acetylcholinesterase (AChE) and butyrylcholinesterase (BChE), as well as other gene products, and are known to increase the risk of several diseases, thus shortening life-expectancy. Some examples of affected enzymes are the ‘atypical’ BChE, which bears the D70G mutation that confers acute sensitivity to anti-cholinesterases [Loewenstein-Lichtenstein Y. et al. (1995) Nat. Med. 1:1225-1226; Neville L. F. et al. (1990) J. Biol. Chem. 265: 20735-20738], and, as mentioned previously, PON1 and its polymorphic variants.

Less dramatic differences in BChE properties than those conferred by the “atypical” mutation are conferred by the K-variant, which involves an A539T substitution in BChE and reduces the catalytic activity by 30% [Bartels C. F. et al. (1992) Am. J. Hum. Genet. 50:1086-1103]. This variant has an allele frequency of 12% in the Caucasian population. This incidence is therefore high enough to test for the association of BChE-K with the risk of developing specific diseases. This was first attempted with regard to Alzheimer's disease (AD), where some [Lehmann D. J. et al. (1997) Hum. Mol. Genet. 6:1933-1936; Lehmann D. J. (2000) Hum. Genet. 106:447-452.] but not all [Brindle N. et al. (1998) Hum. Mot. Genet. 7:933-9351 reported an increased risk for late-onset AD in BChE-K carriers. Yet more recently, BChE-K's association with coronary artery disease (CAD) was tested. [Nassar (2002) id ibid.] The outcome of that study suggested an increased risk for CAD in BChE-K carriers, as patients with early onset CAD had greater BChE-K frequency than patients with the late-onset disease. Although that difference was not significant, patients with early-onset CAD were significantly more likely to carry both BChE-K and the ε4 allele of apolipoprotein ε, which by itself increases the risk for early-onset CAD [Premkumar D. R. (1996) Am. J. Pathol. 148:2083-2095]. The hypothesis was advanced that this reflects additive risks from these polymorphisms for the development of premature CAD [Nassar (2002) id ibid.). The current evidence thus suggests a synergism between the effects of BChE and apolipoprotein ε, which has been proposed to protect both the cardiovascular system and the CNS from oxidative stress, and its ε4 variant of apolipoprotein ε is apparently less effective in this function [Nassar B. A. et al. (1999) Clin. Biochem. 32:275-282), leading to enhanced risk for both AD and CAD.BChE has indeed been reported to interact with lipoproteins and alter their metabolism. [Abbott C. A. et al. (1993) Clin. Sci. (Lond) 86:77-81]. Therefore, the combination of two variants with insufficient protective capacities may cause more significant cumulative damage to the cardiovascular and nervous systems. The related effect of paraoxonase polymorphism, AChE and lipoprotein ε4 are not the only proteins, the functions of which intersect that of BChE. As mentioned before, PON1 also participates in detoxification of organophosphates [Masson P. et al. (1998) J. Physiol. Paris 92:357-362], and prevents lipoxidation of LDL and HDL. [Mackness B. (1998) id ibid.]. A decreased PON1 level obviously places an additional burden on the detoxifying function of BChE, especially under the stress of organophosphate exposure. However, although the PON1 R allele has been reported to selectively increase the risk for CAD [Adkins S. et al. (1993) Am. J Hum. Genet. 52:598-608], there appeared to be no interaction between BChE-K and PON1-R in increasing this risk [Nassar (2002) id ibid.]. It is therefore possible that BChE-PON1 interactions would be relevant only in those diseases that develop following OP exposure, but not in every case of CAD.

The present invention stems from the inventors' findings that, amongst the PON1 and the ACHE polymorphisms, there seems to be a tendency of certain alleles to segregate together. Interestingly, the resulting combination of PON1 and ACHE alleles and the incidence of PD in the carriers suggest that there is an haplotype which is more susceptible to insecticide induced PD.

Hence, the present invention has as an object utilizing such haplotype as a diagnostic tool for evaluating the risk of PD, both individually and for the population of interest. This and other objects of the invention will be elaborated on as the description proceeds.

### SUMMARY OF THE INVENTION

The present invention relates to the use of the “Parkinson Disease (PD)-susceptibility haplotype”, as herein defined, as a tool for the prediction of PD risk and severity in a population and/or an individual subjected to environmental exposure to anticholinesterase(s).

In particular, the invention relates to a method of predicting genetic predisposition to PD, by the following screening method:
(a) providing a blood sample from an individual to be screened; and
(b) analyzing the DNA from said blood sample for the presence or absence of the “PD-susceptibility haplotype” as herein defined, by appropriate means; whereby the presence of the “PD-susceptibility haplotype” indicates a higher predisposition to PD, and the absence of the “PD-susceptibility haplotype” indicates a lower predisposition to PD, compared to a control.

Therefore, in another aspect the present invention provides a method of testing a blood sample of a human subject for the presence of the “PD-susceptibility haplotype”, by analyzing the DNA of said blood sample by appropriate means, wherein the presence of the “PD-susceptibility haplotype” indicates a higher predisposition of said human subject to PD, and the absence of the “PD-susceptibility haplotype” indicates a lower predisposition of said human subject to PD, compared to a control.

In yet a further aspect, the invention relates to a kit for screening for genetic predisposition which essentially comprises means for collecting blood samples and for isolating DNA therefrom and reagents for detecting the presence of the said “PD-susceptibility haplotype”.

The invention will be described in more detail on hand of the following Figures.

### BRIEF DESCRIPTION OF THE FIGURES

FIG. 1: The PD susceptibility locus on Chromosome 7. Spanning the region of 9_{21.8}-9₂₂ on the long arm of chromosome 7, this locus includes the PON, ACHE, ARS, and PIX genes [Grant et al. (2001)].

Abbreviation: centrom., centromere.

FIG. 2: PON1 gene polymorphisms.

Abbreviation: regul. reg., regulatory region; st.si., start site; cod. reg., coding region.

FIG. 3: Schematic of the AChE gene.

FIG. 4a-b: AChE gene polymorphisms

FIG. 4a: Incidence of the deletion mutation on the HNF3 binding site on the AChE promoter region. The frequency in the Israeli population is ten-fold higher, when compared to the U.S.A. This frequency is based on our screening of North Carolina volunteers as compared with Israelis [Shapira (2000) supra]. It is further compatible with the 80% linkage which was found between the HNF₃ mutation and the H322N polymorphism in Israelis [Shapira (2000) supra] and with the relatively high incidence of this H322N polymorphism, which determines the Ytb blood group in middle-east population [Ehrlich et al. (1994) Genomics 22(2): 288-95]. The linkage between the different polymorphisms on this locus extends throughout the ACHE site and showed 100% linkage between the “silent” P446 polymorphism [Bartels et al. (1993) supra]. Thus, the HNF₃ deletion, the H322N mutation and the silent. P446 polymorphism are all linked together, creating a haplotype.

FIG. 4b: AChE polymorphisms, described by Shapira (2000) and Bartels (1993) [Shapira M. et al. (2000) Hum. Mol. Genet. 9:1273-1281; Bartels C. F. et al. (1993) J. Hum. Genet. 52:928-936].

Abbreviations: freq., frequency.

FIG. 5: BChE activity in urban and rural PD patients.

The graph shows specific BChE activity in nmoles/min/mg protein. The activity of BChE in the group of exposed is lower than in non-exposed subjects, irrespective of the presence of the mutation. This proves exposure risk in the examined population.

Abbreviations: Act., activity; urb., urban; rur., rural.

FIG. 6: Serum BChE activity.

The graph shows specific BChE activity in nmoles/min/mg protein in urban and rural groups, showing lower BChE activity in the rural PD group compared to urban. No significant differences were found between carriers (carr) and non-carriers (wt). Numbers in each column indicate sample size in each group. This proves validity of comparison between Rural and Urban populations.

Abbreviations: Act., activity; Ser., serum; urb., urban; rur., rural.

FIG. 7: Serum AChE activity.

The graph shows specific AChE activity in nmoles/min/mg protein in urban and rural groups. Lower AChE activity in the rural compared to urban. No significant differences were found between carriers (carr) and non-carriers (wt). Numbers in each block indicate sample size in each group. p=0.03

Abbreviations: Act. activity; Ser., serum; urb., urban; rur., rural

FIG. 8: AChE activity in PD patients.

AChE activity in PD patients with mutations is lower than without mutations. The differences are statistically significant, p=0.003.

Abbreviations: Healt., healthy; rur., rural; Park., Parkinsonian; Act., activity.

FIG. 9: BChE activity in PD patients.

BChE activity in PD patients with or without mutations is lower than rural healthy individuals.

Abbreviations: Healt., healthy; rur., rural; Park., Parkinsonian; Act., activity.

FIG. 10: The ACHE promoter polymorphism in various age groups and health conditions in Israel.

Abbreviations: inc., incidence; mut., mutation; S., sick; H., healthy.

FIG. 11: Percent frequency of HNF mutation.

Abbreviations: cont., control; pat., patients; n.-exp., non-exposed; pre-exp., pre-exposed.

FIG. 12: The PON1-ACHE polymorphism pattern.

Abbreviations: Chr., chromosome.

FIG. 13a-b: The analyzed genotypes.

FIG. 13a: Shown are the chromosome position and the polymorphic sites that were studied in the PON1 [GenBank Accession Number AF539592] and ACHE [GenBank Accession Number AF002993] genes. Nucleotide numbers begin with the translation start site at 0. Shown below in italics are the biological effects of the polymorphisms. Nucleotides associated with the rare haplotype are noted below.

FIG. 13b: Shown is the linkage disequilibrium analysis of the tested polymorphisms, presented as absolute r and D′ values in parallel matrices. Note the appearance of apparently linked polymorphisms, highlighted in blue.

Abbreviations: link., linkage; del., deletion; hypersens., hypersensitivity; M.E. freq., Middle East frequent.

FIG. 14: Over-representation of ACHE deletion, but not of PON1 polymorphisms in exposed PD subjects.

Allele frequencies of the ACHE promoter deletion and PON1 coding sequence polymorphisms are shown. Numbers (n) involved for ACHE and PON polymorphisms in the general population, PD non-exposed, and PD exposed groups: 464/287, 59/40 and 39/34, respectively.

Abbreviations: Incid., incidence; Gen. Pop., general population; n.-exp., non-exposed; exp., exposed.

FIG. 15a-c: Reduced serum activities of AChE and PON, but not BChE or arylesterase in PD patients.

FIG. 15a: Serum cholinesterases activities in the general population compared to PD patients.

FIG. 15b: PON1 and arylesterase activities in the general population compared to PD patients.

FIG. 15c: Average of the specific activities of cholinesterases in PD polymorphism carriers and non-carriers.

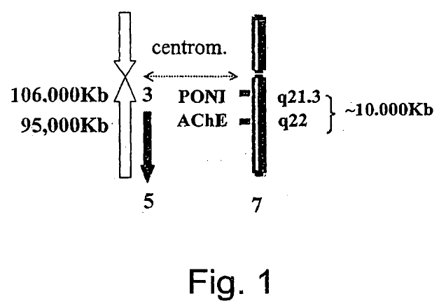

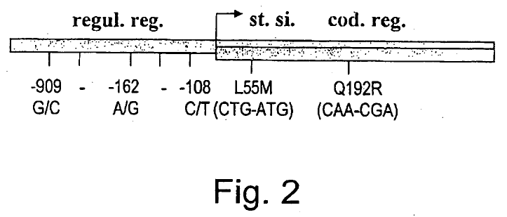

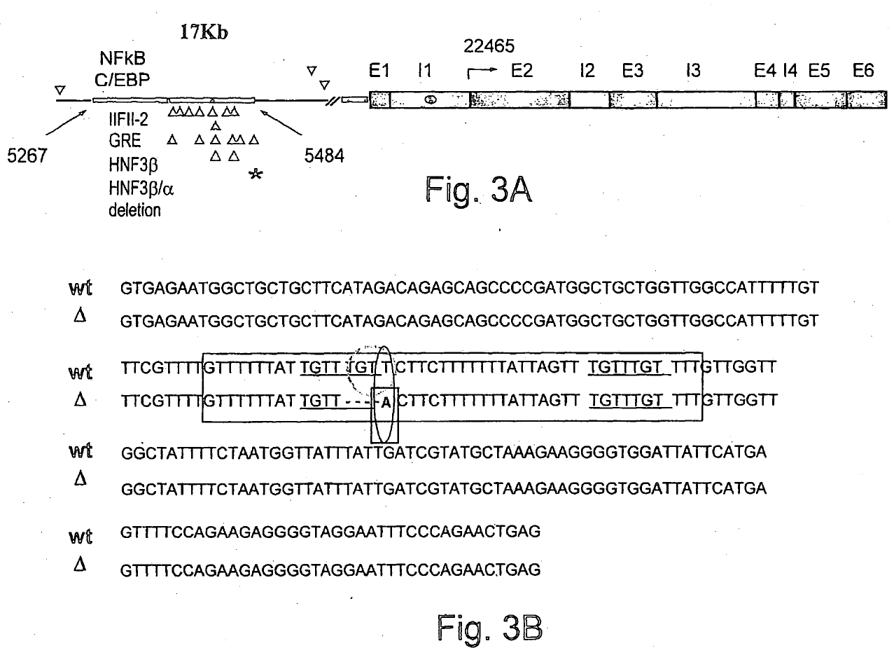

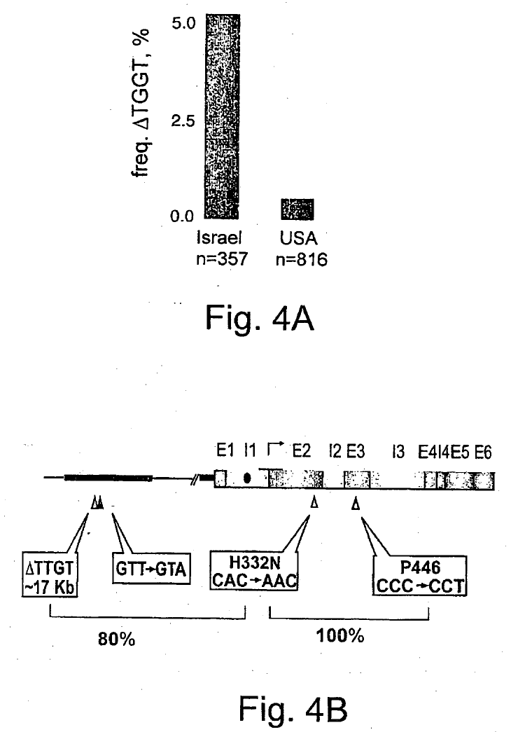

Abbreviations: Spec. Act., specific activity; Gen. Pop., general population; Aver. Spec. Act., average specific activity.

### DETAILED DESCRIPTION OF THE INVENTION

The present invention relates to a haplotype present on human chromosome 7, which the inventors have found to be directly linked to a higher susceptibility to develop Parkinson's Disease (PD).

This haplotype is comprised by the presence of PON1 alleles L55M, Q192R and ACHE alleles del HNF3, H332N, P446 in linkage, demonstrated by an apparent segregation frequency of 100% in PD patients, which is significantly different from the expected frequency of segregation for unlinked alleles of 5%. Thus, the inventors named this the “Parkinson's Disease (PD)-susceptibility haplotype” which is associated with increased risk to develop this neurodegenerative disease, especially following exposure to anticholinesterases.

Thus, in a first aspect, the present invention relates to the use of the “PD-susceptibility haplotype” as a tool for the prediction of PD risk and severity in a population and/or an individual.

As shown in the Examples, the PD-susceptibility haplotype is present in 9% of the examined PD patients. It is characterized by having alleles L55M and Q192R of PON1 segregating in linkage with alleles del HNFs AND H332N AND P446 of AChE. This is not a random event since the two genes are 5.5 megabase apart, indicating independent recombination.

In a second aspect, the invention relates to a diagnostic method of predicting susceptibility to PD, based on the detection of the “PD-susceptibility haplotype” in an individual.

Besides the classical method of detection of these polymorphisms, restriction fragment length polymorphism RFLP), newer methods utilize the power of PCR amplification together with the enhanced size resolution by electrophoresis of labeled PCR products on thin polyacrylamide gels. When these techniques are combined in the automated sequencer, accurate sizing and quantification is possible. This technique employs the use of a forward primer, 5′-labeled with the fluorescent dye 6-FAM, and an unlabeled reverse primer in a PCR reaction. The resulting PCR product has a forward primer-derived strand that is labeled and therefore detectable in the ABI PRISM 3700 DNA Analyzer. The automated machine is capable of simultaneously detecting up to four different fluorescent dyes, this allows the use of a fluorescent internal size standard that can run in the same lane as the PCR sample. The use of this internal size standard overcomes lane to lane variation and allows consistent quantification and sizing of PCR products in different lanes.

Single Nucleotide Polymorphism

Detection of single base variations in DNA in the form of a point mutation or single nucleotide polymorphisms (SNP) can serve as a powerful genetic mapping tool. These variations are used to provide insight into population dynamics or pharmacogenomics or to signify phenotypic consequence.

Single nucleotide primer extension is a straightforward method for validation or comparative genotyping of known SNPs and point mutations. This technique permits exact base identity determination of a polymorphic locus without direct sequencing. The information potential of a base change can be effectively determined using the SNaPshot™ ddNTP Primer Extension™ method, which is ideal for locus validation and subsequent screening of individuals (genotyping).

The 3′ terminus of an unlabeled oligonucletoide primer is extended by a single fluorophore-labeled ddNTP; Because the primer is designed to anneal directly adjacent to the variant base of interest and the reaction does not include dNTPs, incorporation occurs only at a single site. Each of the four possible dye labeled terminators in a SNaPshot ddNTP primer extension reaction is labeled with a different rhodamine-type fluorescent dye. The labeled primer extension products are detected and analyzed by the ABI PRISM 3700 DNA Analyzer.

Mutation Detection using the LightCycler

The primary advantages of the LightCycler (LC) are to reduce the time taken for PCR amplification reactions, and to perform semi-quantitative and quantitative RT-PCR analyses. The machine achieves this by using a thin-walled glass capillary, heated and cooled in a stream of air. The physical properties of the capillaries permit extremely rapid heating and cooling of samples. This enables PCR cycling through the stages of denaturation, annealing, and extension to occur at a faster rate than conventional PCR engines. It also eliminates the electrophoretic separation stage of classical PCR.

In addition to being a useful quantitative real-time PCR device, an entirely separate capability of the LC is the detection of known polymorphisms. The LC can measure fluorescent at several different wavelengths. The use of two different fluorescent dyes, fluorescein and Red 640, enables polymorphism detection. The LC can excite fluorescein, which will then emit visible light (fluorescence) at a longer wavelength. If the second dye (Red 640) is in close proximity to the fluorescein, energy transfer occurs, where energy emitted from the excited fluorescein in turn excites the Red 640 dye, which, then produces a secondary emission at 640 nm. This energy transfer process is called fluorescence resonance energy transfer (FRET). The LC can detect the specific fluorescence of the Red 640 dye, and can thus measure the level of fluorescein and Red 640 that are in close proximity to one another.

The LC uses this process to detect polymorphisms by attaching each of the two dyes to different hybridization probes. One probe is longer than the other, and is labeled 5′ with the Red 640 dye. This probe is complementary to a target sequence that is downstream from the mutation site. The other probe is shorter and is complementary to the wildtype sequence of the mutation site. The shorter probe is labeled 3′ with fluorescein. After the target sequence area has been amplified during PCR, a melting curve analysis is performed by gradually increasing the temperature in the capillary. At low temperatures both probes will anneal to the target area, the two dyes will be in close proximity to one another and FRET can occur.

The LC can detect the resulting fluorescence emission from the Red 640 dye on the longer probe. As the temperature increases, the shorter probe will melt away from the target sequence before the longer probe. At this moment FRET will no longer occur, and the LC will detect the subsequent drop in fluorescence at 640 nm. Since the shorter probe is complementary to the wildtype target sequence, any mutation in the target sequence will decrease its affinity for the probe, seen as an decrease in the temperature at which the probe and target dissociate. A drop in the temperature at which FRET is lost is thus indicative of a mutation.

In a third aspect, the invention relates to a method of screening for a genetic predisposition to PD, wherein said method involves the steps of:
(a) obtaining a blood sample from an individual to be screened; and
(b) analyzing the DNA from the blood sample of (a) for the presence or absence of the “PD-susceptibility haplotype” by appropriate means;
   wherein the presence of the “PD-susceptibility haplotype” indicates a higher predisposition to PD, and the absence of the “PD-susceptibility haplotype” indicates a lower predisposition to PD, compared to a control.

Therefore, in another aspect the present invention provides a method of testing a blood sample of a human subject for the presence of the “PD-susceptibility haplotype”, by analyzing the DNA of said blood sample by appropriate means, wherein the presence of the “PD-susceptibility haplotype” indicates a higher predisposition of said human subject to PD, and the absence of the “PD-susceptibility haplotype” indicates a lower predisposition of said human subject to PD, compared to a control.

In a last aspect, the invention provides a kit for screening for a genetic predisposition to PD, including
(a) means for collecting a blood sample;
(b) reagents for detecting the presence of the “PD-susceptibility haplotype”.

Insecticides are detoxified from the organism in three stages. In the first stage, the enzymes p450 (CYP450) and PON1 are involved. In the second stage, the toxic metabolites are conjugated to glutathione transferase, and in the third stage they conjugate to substances that are bound to leave the cell [Ecobichon D. J. and Joy, R. M. (1994) Pesticide and neurological disease 2^{nd}. Ed. CRC Press, Boca Raton; Hodgson E. and Lewy, P. E. (1996), Environ. Health Perspect. 104:97-106]. Genetic polymorphisms have been found in the genes encoding all these enzymes [Menegon (1998) id ibid.]. The present study shows a high incidence of the L55M polymorphism of PON1 in PD patients. The high incidence of the ACHE promoter deletion, the ACHE coding sequence Ytb blood group, and the silent mutation on P446 in ACHE suggests the presence of a defective haplotype in the Mediterranean population, suggesting a genetic basis for the degenerative process triggered by PD.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of Techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

All the study was conducted in accordance with the Provisions of the Committee for Human Trials (Helsinki Committee, Soroka Medical Center, Beer Sheva and Herzog Hospital, Jerusalem, Israel).

A. Population

Three populations were examined:
1. PD patients without chronic exposure to organophosphates (OPs), i.e. urban residents, n=15.
2. PD patients who used to work in agriculture in the past, and most probably chronically exposed to OPs, i.e. kibbutz residents and farmers, n=15.
3. Non-PD (or any other CNS associated disease) population, age and sex comparable, that worked in agriculture, n=10.

All subjects provided informed consent, fllled a medical questionnaire and underwent neurological tests.

B. Anamnesis (Medical History) and Medical Tests

Participants were required to fill in a ethnical data questionnaire including: ethnical background, diseases, past hospitalizations, pharmaceutical treatments, work-related details, residence and also past experiences of exposure to poisons in general and OPs in particular.

C. Collection of Blood Samples

4 ml of blood was obtained from each participant, and collected in EDTA-containing tubes. The blood sample was divided in 300 ml Eppendorf tubes and kept at −75° C. One aliquot was centrifuged at 10000 rpm/30 min. at room temperature (RT) for obtaining serum, which was tested for enzymatic activity.

BChE and AChE Activity

BChE activity in the serum was measured through spectrophotometry, by calculating nmoles of Butyrylcholine (which served as a substrate) degraded/ml serum/time unit as described [Goldsmith J. R. et al. (1990) Arch. Environ. Health 45: 88-94].

AChE activity in serum was measured likewise, using Acetylthiocholine as a substrate.

For measuring both BChE and AChE activities, inhibitors were used in order to inhibit non-specific activity. BW284C51 was used as an inhibitor for AChE, while iso-OMPA was used as an inhibitor for BChE, both at 5×10⁻⁵ M.

D. Quantitative Measurement of Serum Protein

Serum protein was measured by the Lowry method (BioRad Laboratories).

E. DNA Extraction and Detection of Polymorphisms

DNA was obtained from whole blood with Puregene isolation kit (Gentra, Minneapolis). DNA from the AChE gene promoter region was amplified using PCR, and the nucleotide sequence corresponding to the polymorphism site was tested by ABI 3700 in the DNA analysis service unit at the Hebrew University of Jerusalem. Detection of either the A/T substitution, localized in the glucocorticoid receptor binding site, or the 4-nucleotide deletion in the HNF3 binding site, both found in the ACHE promoter region, were performed by sequence analysis [Shapira et al. (2000) id ibid.], or by LC as detailed above.

F. Haplotype Detection

There are two known polymorphisms in the coding region of the AChE gene. One results in an asparagine for histidine substitution on position 322 (H322N), also known as Ytb, and the second is a silent mutation on codon 446 (P446). The. deletion in the promoter region was found in linkage with the H322N mutation by Shapira [Shapira et al. (2000) id ibid.]. In contrast, this same H322N substitution was found by Lockridge [Lockridge O. and Masson P. (2000) Neurotoxicology 21:113-26] to be in linkage with the silent mutation in P446. As a result, the four known polymorphs create a variety of haplotypes in the AChE gene. The inventors tested whether the cariers of the polymorphic gene inherited it from an ancient common origin, as a result of the founder effect, and if so, what was the ethnical origin.

G. Statistical Analysis

Accepted statistical analyses were used, such as ANOVA (single factor), Student's t test (paired, two-tailed), SEM, and correlation test.

### Example 1

A. Anamnestic Data and Clinical Parameters

The anamnestic data and clinical parameters of the subjects are summarized in Tables 3, 4, and 5.

TABLE 3
PD patients living in urban area (Group 1)

| | | Age | | | | | | |
|---|---|---|---|---|---|---|---|---|
| | History | of | | | | | | |
| No., Age, Sex | of | PD | | F. H. | | | | |
| Ethnic origin | A. W. | onset | Medical history | PD | Chronic treatment | Rigid. | Brad. | Trem. |
| 1. 60, F, Poland | − | 62 | Essential | − | Sinemet Gastro | − | − | + |
| | | | tremor, HTN | | | | | |
| 2. 85, M, Poland | + | 78 | CIHD | − | Dopicar Adalat | + | + | + |
| | | | | | Pergolide | | | |
| 3. 85, M, Poland | + | 75 | CIHD, Bed ridden | − | Dopicar Pergolide | + | + | + |
| 4. 72, F, Poland | + | 70 | HTN | − | Dopicar Sinemet | + | + | + |
| 5. 85, F, Germany | + | 75 | — | − | Dopicar Norvasc | − | − | + |
| 6. 88, M, Romania | + | 72 | Bed ridden | − | Dopicar Phenergan | + | + | − |
| | | | dementia | | | | | |
| 7. 73, M, Hungary | + | 63 | — | − | — | + | − | − |
| 8. 82, F, Romsnia | + | 76 | HTN | − | Norvasc Simovil | − | − | + |
| 9. 76, F, Poland | − | 68 | NIDDM | − | Deralin | − | − | + |
| 10. 77, M, Brazil | + | 67 | HTN, Sick sinus | − | Dopicar Sinemet | + | + | + |
| | | | syndrome | | Cartia | | | |
| 11. 76, F, Egypt | + | 77 | Cataract | − | Lipidal Gastro Vitamin D | − | − | + |
| 12. 65, F, Brazil | − | 62 | HTN, Writer cramp | − | Deralin Captopril | + | − | + |
| | color | | | | Fossalan | | | |
| | manuf. | | | | | | | |
| 13. 55, M, Brasil | + | 52 | Meningioma | − | Tegretol Selegiline | + | + | + |
| | | | | | Evitol | | | |
| 14. 68, F, Irak | − | 60 | Dementia | − | Dopicar | + | + | − |
| 15. 69, F, Brazil | − | 60 | HTN, CVA | − | Eltroxin Aspirin | + | + | + |

Abbreviations:
color manuf., color manufacture;
F. H. PD, Family History of PD;
Rigid., Rigidity;
Brad., Bradikinesia;
Trem., Tremor;
History of A. W., History of Agriculture Work

TABLE 4
PD patients living in rural area (Group 2)

| | History | Age | | | | | | |
|---|---|---|---|---|---|---|---|---|
| | of | of | | | | | | |
| No., Age, Sex, | OP | PD | Medical | F.H. | | | | |
| Ethnic origin | exposure | onset | history | PD | Chronic treatment | Rigid. | Brad. | Trem. |
| 1. 73, F, Romania | LOW LEVEL EXPOSURE | 72 | CVA, | − | Glibetic Tamoxil | + | + | − |
| | | | NIDDM | | | | | |
| 2. 72. F, Romania | | 71 | Bed | − | Dopicar Capotan | + | + | + |
| | | | ridden | | | | | |
| | | | HTN | | | | | |
| 3. 76, F, Russia | | 67 | HTN | − | Dopicar Sinemet | − | − | + |
| 4. 67, F, Libian | | 60 | HTN, | − | Dopicar Ropinirol | + | + | − |
| | | | CIHD | | | | | |
| 5. 52, M, Morocco | | 40 | — | + | Dopicar Sinemet | + | + | + |
| | | | | sister | Pergolide | | | |
| 6. 58, M, Argentina | | 52 | — | + | Dopicar Sinemet | − | + | + |
| | | | | father | | | | |
| 7. 72, M, Romania | | 70 | HTN | − | Dopicar | + | − | − |
| 8. 74, M, Russia | | 71 | — | − | Dopicar Sinimet | + | + | + |
| 9. 78, F, Russia | | 72 | CIHD | − | Dopicar | + | + | + |
| 10. 65, M, Russia | | 60 | — | − | Dopicar | + | + | + |
| 11. 70, M, Morocco | | 65 | CIHD | − | Dopicar | ++ | + | − |
| | | | CVA Bed | | | | | |
| | | | ridden | | | | | |
| 12. 78, M, Russia | | 72 | HTN | − | Convertin | + | + | − |
| | | | CIHD | | Normiten | | | |
| | | | | | Dopicar | | | |
| 13. 77, F, Russia | | 72 | HTN, | − | Glibetic Dopicar | + | + | + |
| | | | CVA, | | Aspirin | | | |
| | | | NIDDM | | | | | |
| 14. 68, M, Russia | | 62 | — | + | Dopicar Pergolide | + | + | − |
| | | | | Sister | Comtan | | | |
| 15. 84, M, Poland | | 78 | HTN | − | Dopicar | + | − | + |
| | | | | | Bromocriptine | | | |

Abbreviations:
F. H. PD, Family History of PD;
Rigid., Rigidity;
Brad., Bradikinesia;
Trem., Tremor

TABLE 5
Healthy rural people (Group 3)

| | | | Ethnical | Medical | Permanent | | | |
|---|---|---|---|---|---|---|---|---|
| No. | Age | Sex | origin | history | treament | Rigidity | Tremor | Bradikinesia |
| 1. | 73 | F | Poland | CIHD | Normiten | − | − | − |
| 2. | 78 | M | Poland | HTN | Convertin | − | − | − |
| 3. | 77 | F | Romania | Dermatitis | Ung.Polycutan | − | − | − |
| 4. | 74 | F | Russia | HTN | Simovil | − | − | − |
| | | | | | Normiten | | | |
| | | | | | Cartia | | | |
| 5. | 79 | F | Russia | PAF | Coumadin | − | − | − |
| 6. | 74 | F | Romania | HTN | Lasix | − | − | − |
| | | | | | Verapamil | | | |
| 7. | 70 | F | Hungary | CIHD | Dilantun | − | − | − |
| | | | | | Cartia | | | |
| 8. | 75 | M | Brazil | Asthma | Ventolin | − | − | − |
| | | | | CIHD | Aerowent | | | |
| 9. | 76 | M | Romania | HTN | — | − | − | − |
| 10. | 73 | M | Romania | HTN | Convertin | − | − | − |

Average age was 74.9±9.1 in group 1, 70.9±7.9 in group 2 and 74.9±2.5 in Group 3 (p=0.21). No significant effect was found in the age of onset of PD symptoms.

Two thirds of the subjects of Group 1 reported a short term earlier work in agriculture. In Group 2 there was no history of severe exposure, except that their place of residence and occupation are connected to agriculture, thus it is assumed that these subjects were chronically exposed to OP. There was no significant difference between Groups 1 and 2 with regards to progress and symptoms of the PD. The clinical evaluation seems to suggest that in Group 2, the expression of all 3 components of PD (bradikinesi, rigidity and tremor) was more prominent regarding patient decline in function.

Three participants of the study, all from Group 2, reported a family history of PD. All the patients received Dopical (Levidopa, Carbidopa).

The medications taken by the subjects have no extra epidermal or AChE inhibitory activity, and it is not unreasonable to assume that they would cause the PD symptoms. Details of the drugs constituents are shown in Table 6.

TABLE 6
Drugs and constituents

| Chemical group | Drug | Constituents |
|---|---|---|
| H2 blocker | Gastro | Famotidine |
| L-Dopa + Carbidopa | Dopicar | Levodopa, |
| | | Carbidopa |
| Beta blocker | Adalat | Propranolol |
| Dopamine agonist | Pergolide | Pergolide mesylate |
| Ca channel blocker | Norvasc | Amlodipine |
| | | mesylate |
| Sedatives | Phenergane | Promethazine |
| | | hydrochloride |
| Statins | Simovil | Simvastatine |
| Beta blocker | Deralin | Propranolol |
| | | hydrochloride |
| NSAID | Cartia | Acetylsalicylic acid |
| Statins | Lipidal | Simvastatin |
| ACE-inhibitor | Captopril | Captopril |
| Biphosphonates | Fosalan | Alendronic acid |
| Antiepileptic | Tegretol | Carbamazepine |
| Neuroprotector | Selegiline | Selegiline |
| | | hydrochloride |
| Vitamin | Evitol | Alpha Tocopheryl |
| | | acetate(vit E) |
| Antidiabetic | Glibetic | Glibenclamide |
| Antiestrogen | Tamoxil | Tamoxifen citrate |
| ACE inhibitor | Capoten | Captopril |
| Dopamine agonist | Ropinirol | Ropinirol |
| ACE inhibitor | Convertin | Captopril |
| Beta blocker | Normiten | Atenolol |
| COMT inhibitor | Comtan | Entocapone |
| Dopamine agonist | Bromocriptine | Bromocriptine |
| Diuretics | Lasix | Furosemide |
| Ca channel blocker | Verapamil | Verapamil |
| | | hydrochloride |
| Antiepileptic | Dilantin | Phenytoin |
| Adrenomimetic beta | Ventolin | Salbutamol |
| II | | |
| Antimuscarinic | Aerowent | Ipratropium |
| | | bromide |
| L-Dopa + carbidopa | Sinemet | Co-Carbidopa |

The frequency of patient's complaints is shown in Table 7. There are no significant differences in the frequency of complaints about different body systems between rural and urban PD patients.

TABLE 7

| | Urban PD | | | Rural PD | | | Healthy | | |
|---|---|---|---|---|---|---|---|---|---|
| | patients (n = 15) | | | patients (n = 15) | | | rural people (n = 10) | | |
| Complaint | alight | mod. | severe | alight | mod. | severe | alight | mod. | severe |
| General ill feeling | 20 | | 20 | 20 | 13.2 | | 20 | | |
| Weakness | 20 | 6.6 | 13.2 | 33 | 20 | | 20 | | |
| Headache | 40 | | | 6.6 | | | 20 | 10 | |
| Muscle weakness | 6.6 | 6.6 | 6.6 | 26.4 | 6.6 | 6.6 | | | |
| Tremor | 6.6 | | 33 | 26.4 | 26.4 | 6.6 | | | |
| Arthralgia | 20 | | 33 | 26.4 | 6.6 | 6.6 | | 10 | |
| Visual | 40 | | 6.6 | 20 | 13.2 | 33 | 10 | 30 | |
| disturbances | | | | | | | | | |
| Night blindness | 33 | | | 26.4 | | 6.6 | | | |
| Abdominal cramps | 20 | 6.6 | | 6.6 | 13.2 | 6.6 | | | |
| Diarrhea | 33 | | | | | | | 20 | |
| Constipation | 33 | | 20 | 20 | 13.2 | 6.6 | 20 | 30 | |
| Urinary retention | 13.2 | | | 13.2 | | | 20 | | |
| Urinary urgency | 13.2 | | | 13.2 | 6.6 | 6.6 | 30 | 20 | |
| Vomiting | 13.2 | | | 6.6 | 6.6 | | 20 | | |
| Sialorrea | 13.2 | | | | 6.6 | | | 20 | |
| Dry mouth | 13.2 | | | 20 | | | | 20 | |
| Rhinitis | 13.2 | | | 6.6 | | | | 10 | |
| Chest pain | 13.2 | | | | 6.6 | | 30 | | |
| Techycardia | 13.2 | | | | 6.6 | | | | |
| Difficulty of | 20 | 6.6 | | | 13.2 | | | 20 | |
| concentration | | | | | | | | | |
| Nervousness | 20 | | | 20 | 13.2 | | | 20 | |

Abbreviations:
mod., moderate.

B. Detection of Polymorphisms

Amongst the 40 subjects studied for polymorphism in the ACHE gene, there were 5 carriers of the HNF3-binding site four-nucleotide deletion, which were 1 from Group 1 and 4 from Group 2. There were no carriers of this polymorphism in Group 3.

This result is consistent with the frequency found in the general population of 1:26. In this study, the frequency is 1:25 in the exposed or non-exposed PD patients, and 1:4 in the chronically exposed PD patients (Group 2).

As in earlier studies, no homozygotes for this polymorphism were found, possibly because bearing two copies of this allele might be lethal. All 5 patients with this deletion were also carriers of haplotype Ytb. This indicates that there is linkage between these two polymorphisms, and likely common genomic origin. In addition, a third ACHE polymorphism, P446 was found in 5 subjects, 4 heterozygotes and 1 homozygote, such that the idea of a common ancient origin of the haplotype is reinforced.

In 12 patients, from Groups 1 and 2, the L55M polymorphism of PON1 was detected. Q192R was also tested. There was no unequivocal linkage between PON1 and ACHE.

There was no correlation in age, sex, age of onset, ethnical origin and medical history between the carriers of the deletion mutation which participated. It is important to indicate that in Shapira [Shapira et al. (2000) id ibid.], there was one carrier of the ACHE HNF3 deletion who was very sensitive to AChE inhibitors and another that had a history of recurring miscarriages. In the women carrying this deletion in the present study no miscarriages were reported, although the fertility age of most of them might have preceded exposure. In addition, it was found that out of five HNF3 carriers, three also carried this mutation in oral epithelial cells as well excluding the possibility that this was a somatic mutation in blood cells. The lack of mutation in the other 2 patients might be the result of technical difficulties regarding the identification of the mutation in the oral epithelial cells, the yield of which was rather low.

C. AChE and BChE Activities

Table 8 summarizes the results of serum enzymatic activity and the mutations. It is important to note that in the tested-population, the number of possible haplotypes is 640 combinations, while in this study only four possible combinations were found, leading to the conclusion that there is a common genetic origin of the tested population.

TABLE 8
AChE and BChE activities (nmol/min per ml or per mg) in
serum, protein concentration, and mutations found in patients' blood

| | Protein | | | | | MUTATIONS | | | |
|---|---|---|---|---|---|---|---|---|---|
| | conc. | AChE | AChE | BChE | BChE | | | | PON1 |
| No. | mg/ml | per ml | per mg | per ml | per mg | HNF | YTb | P446 | (L55-M) |
| 1 | 17.5 | 53.1 | 3.03 | 2955 | 168.8 | − | − | − | − |
| 2 | 22.4 | 29.4 | 1.3 | 2269 | 101.3 | − | − | − | − |
| 3 | 16.7 | 16 | 0.06 | 1038 | 62.1 | − | − | − | + |
| 4 | 15.5 | 22.4 | 1.45 | 1966 | 126.8 | − | − | − | + |
| 5 | 22.3 | 36.7 | 1.65 | 1594 | 71.4 | − | − | − | − |
| 6 | 16.6 | 74.6 | 4.49 | 722 | 43.4 | − | − | − | − |
| 7 | 17.6 | 60.3 | 3.4 | 2729 | 155 | − | − | − | − |
| 8 | 18.7 | 18.9 | 1 | 992 | 53.3 | − | − | − | − |
| 9 | 17.5 | 27.9 | 1.6 | 2072 | 118.4 | + | + | + | + |
| 10 | 12.2 | 32.1 | 2.63 | 1004 | 82.8 | − | − | − | + |
| 11 | 8.1 | 36.5 | 4.5 | 2006 | 247.6 | − | − | − | + |
| 12 | 11.7 | 35.2 | 3 | 2637 | 225.3 | − | − | − | + |
| 13 | 23.6 | 50.3 | 2.13 | 1379 | 58.4 | − | − | − | + |
| 14 | 23.4 | 30.6 | 1.3 | 1698 | 72.3 | − | − | − | − |
| 15 | 12.2 | 30.6 | 2.5 | 1140 | 93.4 | − | − | − | − |
| 16 | 24.5 | 27.5 | 1.12 | 1231 | 50.2 | − | − | − | + |
| 17 | 25.3 | 47.2 | 1.86 | 1443 | 57 | − | − | − | − |
| 18 | 24.6 | 64.1 | 2.6 | 2247 | 91.3 | − | − | − | − |
| 19 | 22.8 | 52.3 | 2.29 | 1948 | 85.4 | − | − | − | − |
| 20 | 22.8 | 59.7 | 2.62 | 2598 | 113.9 | − | − | − | − |
| 21 | 28 | 30.2 | 1 | 1598 | 57 | + | + | + | + |
| 22 | 28.4 | 57.5 | 2 | 1440 | 50.7 | − | − | − | − |
| 23 | 24 | 46.5 | 1.94 | 2165 | 90.2 | − | − | − | − |
| 24 | 26.2 | 31.4 | 1.2 | 1775 | 67.7 | + | + | + | + |
| 25 | 24.6 | 40.3 | 1.64 | 1337 | 54.3 | + | + | + | + |
| 26 | 18.3 | 21.7 | 1.18 | 559 | 30.5 | + | + | + | + |
| 27 | 56.2 | 39.6 | 0.7 | 5008 | 89.2 | − | − | − | − |
| 28 | 23 | 55.4 | 2.4 | 1369 | 59.5 | − | − | − | − |
| 29 | 46.5 | 54.2 | 1.16 | 2498 | 53.7 | − | − | − | − |
| 30 | 20.8 | 35.1 | 1.67 | 1147 | 55.1 | − | − | − | − |
| 31 | 27.9 | 59.4 | 2.12 | 6494 | 232.8 | − | − | n.d. | n.d. |
| 32 | 55.4 | 61.3 | 1.1 | 5821 | 105 | − | − | n.d. | n.d. |
| 33 | 28.4 | 121.2 | 4.27 | 6982 | 245.8 | − | − | n.d. | n.d. |
| 34 | 28.6 | 35.5 | 1.24 | 3953 | 138.2 | − | − | n.d. | n.d. |
| 35 | 35 | 43 | 1.23 | 1678 | 77.9 | − | − | n.d. | n.d. |
| 36 | 22.2 | 63.6 | 2.86 | 4224 | 190.2 | − | − | n.d. | n.d. |
| 37 | 2.28 | 37.9 | 1.66 | 135 | 59.2 | − | − | n.d. | n.d. |
| 38 | 35 | 34 | 0.97 | 4842 | 138.2 | − | − | n.d. | n.d. |
| 39 | 40.2 | 38 | 0.95 | 6706 | 166.8 | − | − | n.d. | n.d. |
| 40 | 32.8 | 61.6 | 1.87 | 5116 | 155.9 | − | − | n.d. | n.d. |

| | | | | |
|---|---|---|---|---|
| FREQUENCY OF MUTATION (%) | 12.5 | 10 | 10 | 27.5 |

Abbreviations:
n.d. = not done.

D. Differences in the AChE and BChE Activities

Table 9 shows the differences in the AChE and BChE activities.

TABLE 9

| | Urban PD patients | | Rural PD patients | | Healthy rural |
|---|---|---|---|---|---|
| | (n = 15) | | (n = 15) | | subjects |
| | wt* (n = 14) | carr** (n = 1) | wt (n = 11) | carr (n = 4) | (n = 10) |
| AChE activity | 2.32 ± 1.3 | 1.6 | 1.85 ± 0.64 | 1.26 ± 0.27 | 1.87 ± 1.04 |
| nmole/min/mg | | | | | |
| BChE activity | 111.5 ± 64.8 | n.d. 118.4 | 72.3 ± 22.1 | 52.3 ± 15.7 | 151 ± 61.19 |
| nmole/min/mg | | | | | |

Abbreviations:
wt = non-carrier of the mutation;
carr = carrier of the mutation.
Results are expressed as mean ± SD.

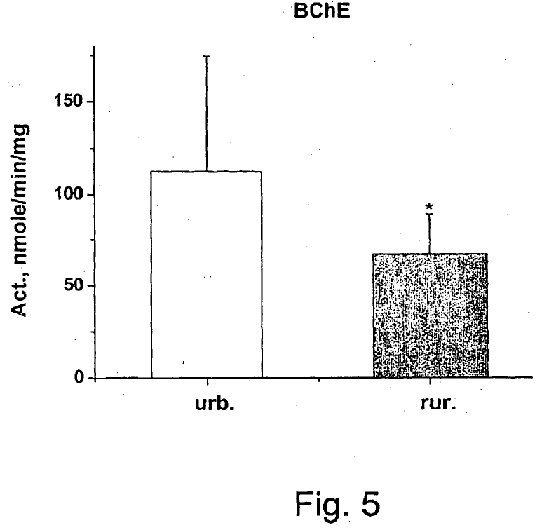

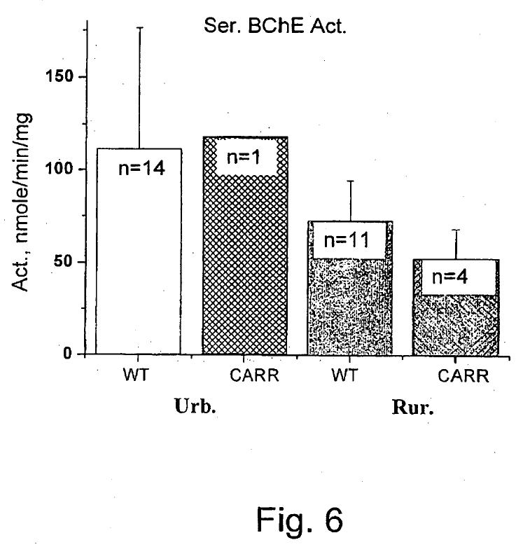

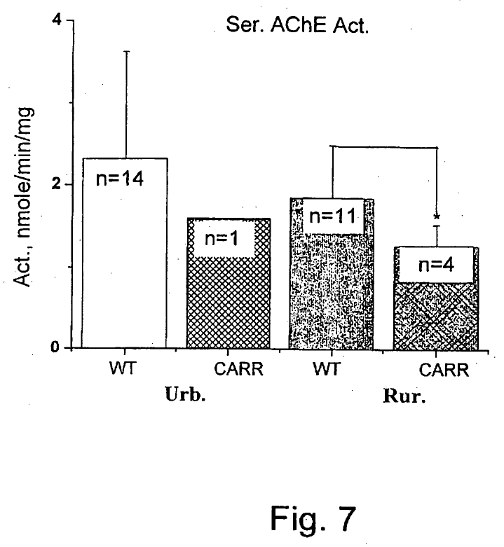

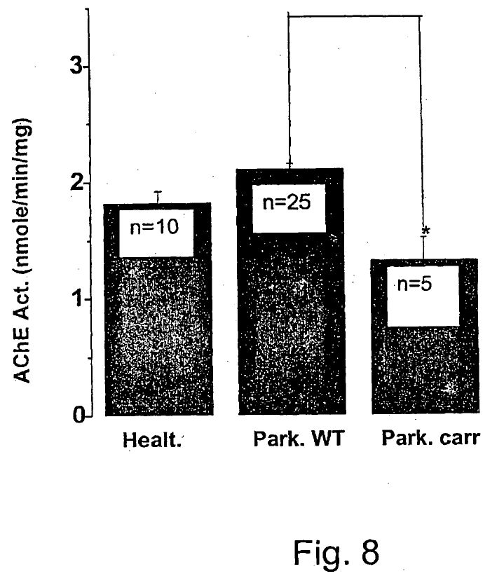

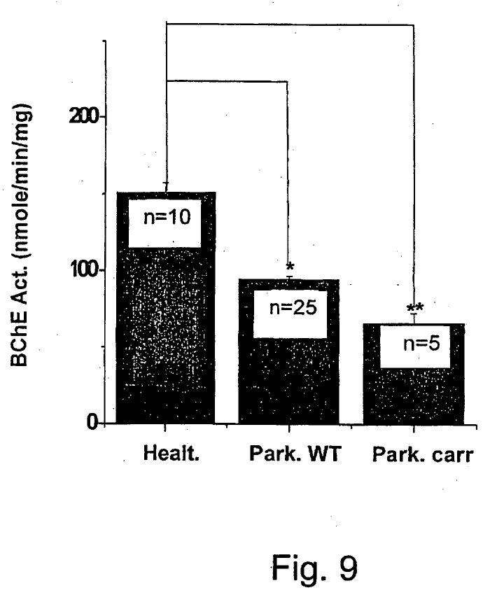

As shown in Tables 8 and 9 and in FIGS. 5 to 9, BChE activity is higher in urban subjects than rural subjects, wherein p=0.02. This is possibly a result of the exposure to PO.

BChE activity in rural PD patients carriers of the HNF3 polymorphism is not statistically different from BChE activity in PD patients non-carrier is, with no connection to the place of residence.

AChE activity in PD patients who are HNF3 carriers is about 50% lower than the activity in non-carrier, rural PD patients. Shapira (2000) reported an increase in AChE in erythrocytes of carriers of the ACHE promoter deletion [Shapira et al. (2000) id ibid.]. It is possible that the carriers of mutations who are chronically exposed to OPs cannot increase AChE blood levels in response to exposure, and due to this failure of protection against stress they might be more sensitive to OP exposure and tend to develop a degenerative process under this exposure.

BChE activity in Group 3 is higher than BChE activity in PD patients both carriers and non-carriers of the HNF3 mutation. The differences are statistically significant, with p=0.003 for carriers and p=0.002 for non-carriers. This difference in BChE activity in the two groups is surprising. Two possible explanations are envisaged. The control group subjects (Group 3) might have been less exposed to OPs, or alternatively, their liver metabolism is more active, leading to more BChE production, perhaps due to the fact that they do not receive routine drug treatments.

### Example 2

Frequency of “HNF-Polymorphism” on the ACHE Promoter

Over one third of PD patients suffer cholinergic deficiencies [Soreq and Zakut (1993) id ibid. J. Exposure to anti-AChEs, which causes AChE over-expression, is known to increase the risk for PD [Kaufer and Soreq (1999) id ibid.]. Therefore, the inventors explored the possibility that the HNF₃ mutation, which activates ACHE gene expression, is also associated with an increased risk for PD. The results of this study indicate that this previously unforseen hypothesis seems to be true.

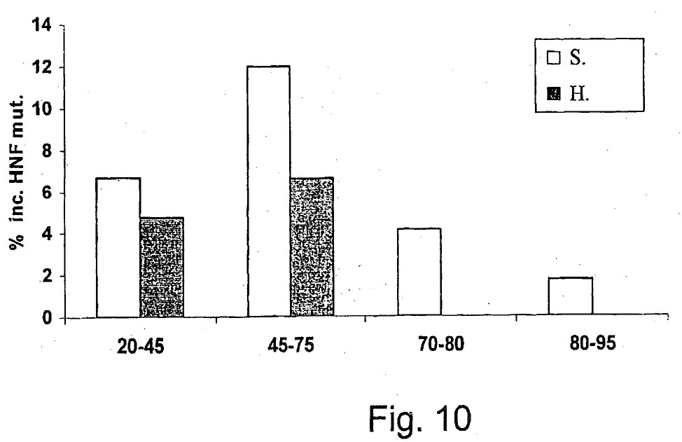

The incidence of ACHE promoter polymorphism was tested in several groups of Israeli individuals, healthy and unhealthy. The latter included women with pregnancy complications, and older patients following stroke or Parkinson's Disease patients (FIG. 10).

Because of the small sample size, the frequency of the mutation in some of the groups must be considered a preliminary finding. The apparently higher incidence of the ACHE promoter polymorphism in elderly patients with Parkinson's Disease initiated further analysis into this potential correlation.

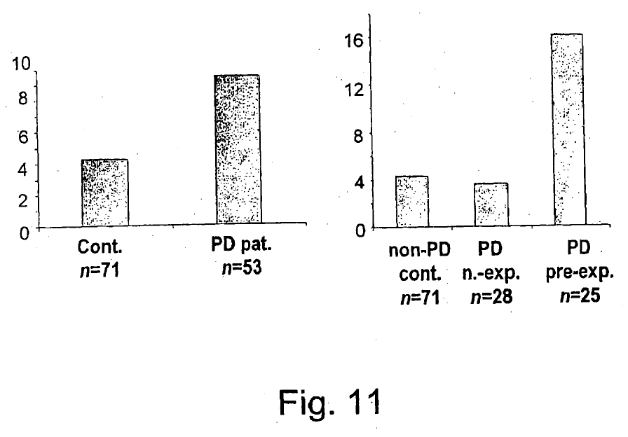

FIG. 11 shows the analysis of the incidence of the HNF polymorphism in ACHE in PD patients, in particular those exposed to agricultural insecticides Indeed PD patients presented a higher frequency of the HNF polymorphism.

The increase in frequency was especially significant in PD patients pre-exposed to agricultural insecticides.

### Example 3

PD Screen of PON1 Alleles in Israeli Versus Other Populations

TABLE 10
Healthy population

| | | | Japan |
|---|---|---|---|
| | | Russia | (Kondo et al. |
| | | (Akhmedova et | 1998^{c}; Suchiro et |
| PON1 allele | Israel | al., 1999^{a}; 2001^{b}) | al., 2000^{d}) |
| 55L | 0.61 | 0.69 | 0.94 |
| 55M | 0.39 | 0.31 | 0.06 |
| 192Q | 0.7 | 0.74 | 0.381 |
| 192R | 0.3 | 0.26 | 0.691 |

^{a}[Akhmedova, S. et al. (1999) Hum Hered 49, 178-180.
^{b}[Akhmedova et al. (2001) id ibid.]
^{c}[Kondo et al. (1998) id ibid.]
^{d}[Suehiro, T. et al. (2000) Atherosclerosis 150, 295-298.]

TABLE 11
PD population

| | | | Japan |
|---|---|---|---|
| | | Russia | (Kondo et al. |
| PON1 | Israel | (Akhmedova et | 1998^{c}; Suchiro et |
| allele | (n = 39) | al., 1999^{a}; 2001^{b}) | al., 2000^{d}) |
| 55L | 0.54 | 0.57 | — |
| 55M | 0.46 | 0.43 | — |
| 192Q | 0.69 | 0.75 | 0.278 |
| 192R | 0.31 | 0.25 | 0.722 |

^{a}[Akhmedova et al. (1999) id ibid.]
^{b}[Akhmedova et al. (2001) id ibid.]
^{c}[Kondo et al. (1998) id ibid.]
^{d}[Suehiro et al. (2000) id ibid.]

TABLE 12
PD patients' genotype in the Israeli population

| | HNF carriers | Non-carriers |
|---|---|---|
| PON1 allele | (n = 5) | (n = 39) |
| 55L | 0.5 | 0.54 |
| 55M | 0.5 | 0.46 |
| 192Q | 0.5 | 0.69 |
| 192R | 0.5 | 0.31 |

Tables 10, 11 and 12 show the results of comparisons of the genotype presented by PD patients in Israel in comparison with Russia and Japan. The healthy population in Israel mimics the Russian but not the Japanese population with respect to the PON1 polymorphism pattern. There are more L55, Q192 carriers than M55, R192 carriers.

Interestingly, the PD population in Israel is also closer to the Russian one, as evidenced by the frequency of polymorphism in both positions 55 and 192.

The HNF mutation carriers among the Israeli PD patients have lower incidence of Q192 allele then healthy subjects (like the Japanese patients, distinct from the Russians).

The present results are still considered preliminary, since the sample size is relatively small.

### Example 4

Study of the genotype of PD patients in Israel revealed a single haplotype which apparently spans over 5,500 Kb and includes both PON1 and ACHE polymorphisms.

TABLE 13
Apparent linkage between ACHE and PON1 polymorphisms

| Patient | Haplotype | Personal details (medical |
|---|---|---|
| No. | 55 - 192 - □ - Yt - P446 | history, sex, country of birth) |
| 35 | L/M - Q/R - het - A/B - het | OP exposure, M, 58, Argentina |
| 40 | L/M - Q/R - het - A/B - hom | OP exposure, M, 78, Russia |
| 41 | L/M - Q/R - het - A/B - het | OP exposure, M, 65, Russia |
| 46 | L/M - Q/R - het - A/B - het | OP exposure, M, 58, Argentina |
| 1027 | L/M - Q/R - het - A/B - het | OP exposure, M, 70, Morocco |
| 1007 | M/M - Q/Q - het - A/B - het | OP exposure, M, 42, n.k. |

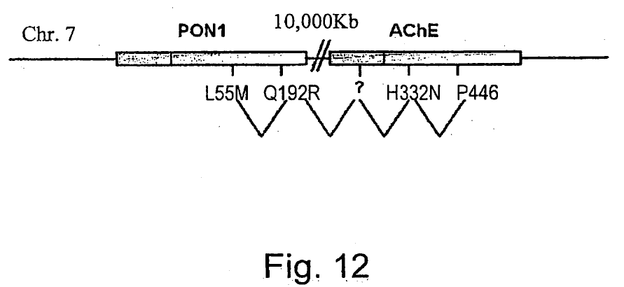

Note that in spite of the different ethnic origins, there is a common genotype that spans both the ACHE and PON1 loci in all PD patients. Thus, the results suggest that risk-associated variants of PON1 and ACHE may be genetically linked. FIG. 12 shows the possible combinations of haplotypes that may exist in the general population. Interestingly, the haplotype including the PON1 55/192 mutations, and the ACHE HNF/yt/P446 polymorphisms, herein referred to as the “PD-susceptibility haplotype”, is the predominant amongst PD patients, suggesting its relevance to causing the disease.

It is important to note that, as mentioned previously, both genes have variants that increase PD risk. Therefore, the combined inheritance of these variants may induce yet a larger risk to PD, and is being tested in a larger population

### Example 5

Linkage Disequilibrium in the ACHE/PON1 Locus

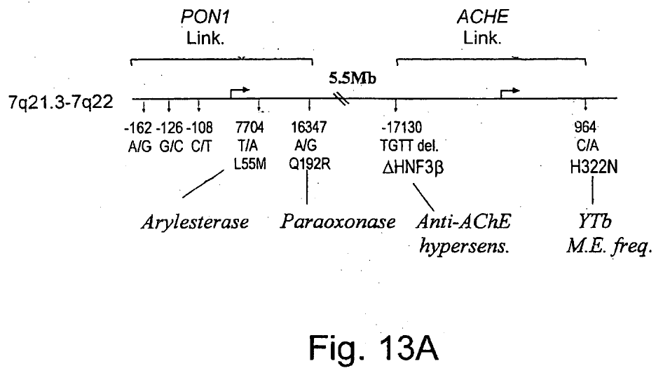

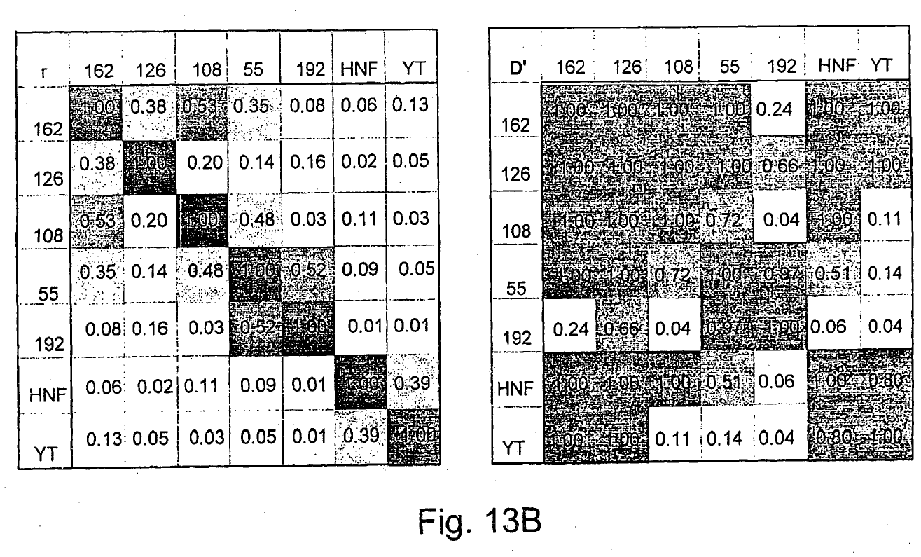

Nucleotide polymorphisms analysis (SNP) was performed on two 7 Kb regions in the PON1 genes of 39 PD patients who live in a rural area that is under routine exposure to insecticides, especially parathion [Herishanu et al. (1989) Can J Neurol Sci 16: 402-5], 59 patients with PD from an urban area with no history of exposure and 454 unrelated disease-free subjects. Seven polymorphic sites and the ACHE promoter deletion were tested: 5 SNPs in the PON gene and 2 SNPs in ACHE, gene (FIG. 13a). Of these, 4 are common SNPs (minor allele frequency>10%) and 3 are rare (minor allele frequency: for the activating ACHE promoter, HNF=2%; the PON1 promoter polymorphism PON126=3%; and the H332N substitution in AChE that yields the YTb blood group phenotype YT=8%). SNP analysis revealed a total of 28 haplotypes in the Israeli population, out of the possible 128. A rare haplotype was identified which includes both the ACHE promoter deletion associated with anticholinesterase hypersensitivity and the enzymatically debilitating PON1 polymorphisms (FIG. 13a, b). Of these, 14 haplotypes appeared in >1% incidence and 8 account for 90% of the variance. Within the PON1 genes, 11 out of the 32 possible haplotypes were observed. Of these, 9 appeared in >1% incidence and 6 account for 90% of the variance. FIG. 13a presents the analyzed locus and FIG. 13b, the linkage equilibrium analysis of the tested polymorphisms.

Linkage disequilibrium analysis demonstrated tight interaction between the ACHE and PON1 genes, 5.5 Mb apart, with distinct representation of specific polymorphisms in these two genes within PD patients as compared with the control population. PD patients presented high incidence of the ACHE promoter deletion associated with the M55 and R192 alleles of PON1 and with the C108 variant in its promoter. In contrast, control individuals carrying the ACHE promoter deletion with the inter-related L55 and R192 PON1 polymorphisms tended to carry T₁₀₈ in its promoter (FIG. 13a, b).

### Example 6

Apparent Association with Exposure-Induced PD Risk

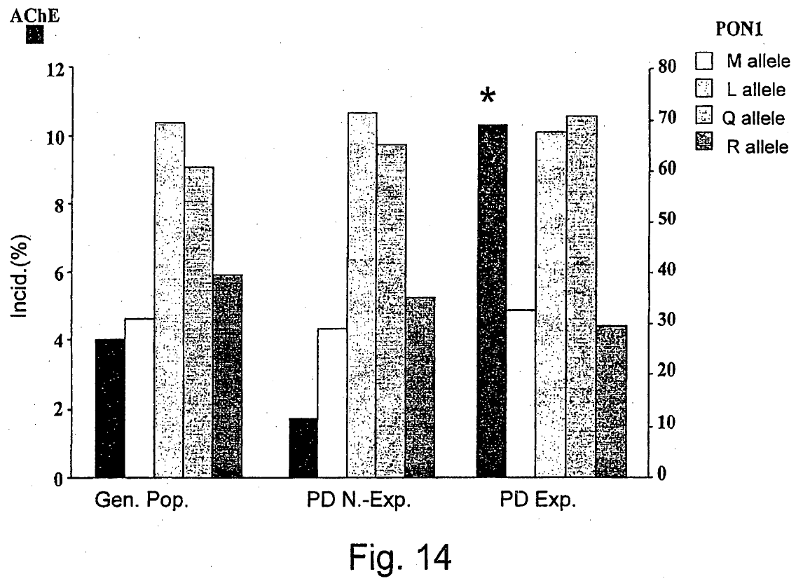

The haplotype composition of insecticide-exposed and non-exposed PD samples did not differ substantially. However, the above rare haplotype was strongly over-represented in the exposed PD samples whereas the ACRE polymorphism associated with anticholinesterase hypersensitivity was under-represented in the non-exposed PD samples, as compared with no-disease controls (FIG. 14; P<5).

Haplotype frequencies were estimated by the EM algorithm [Dempster, A. P. et al. (1977) J Royal Statist. Soc. Ser. B. 39] and tested by likelihood ratio test (LRT) and permutations (n=1000). P values were not corrected for multiple testing.

This analysis was performed for the 3 SNPs that showed modest association with exposed PD (SNPs: 162, 108, HNF), and suggested that the core haplotype determining increased risk for PD, under continued exposure, includes the 108 promoter polymorphism in PON1 as well as the promoter deletion in ACHE (HNF; Tables 14a-c).

Tables 14a-c: Haplotype Analysis.

TABLE 14a
Allele frequencies

| | | | |
|---|---|---|---|
| | PD non- | | PD |
| Haplotype | exposed | Control | exposed |

| | | | | |
|---|---|---|---|---|
| 1 | 1 | 45.0% | 42.9% | 24.2% |
| 1 | 2 | 1.3% | 0.0% | 6.1% |
| 2 | 1 | 53.8% | 55.6% | 69.7% |
| 2 | 2 | 0.0% | 1.5% | 0.0% |

| | | | |
|---|---|---|---|
| N | 40 | 99 | 33 |

| SNPs | Allele 1 frequency | | |
|---|---|---|---|
| 108 | 46.25% | 42.93% | 30.30% |
| HNF | 98.75% | 98.48% | 93.94% |

TABLE 14b
PD exposed vs. PD non-exposed

| | |
|---|---|
| SNPs | P value |

| 162 | 108 | HNF | LRT | Permutations |
|---|---|---|---|---|
| 1 | 1 | 1 | 0.23 | 0.03 |
| 0 | 1 | 1 | 0.04 | 0.03 |
| 1 | 0 | 1 | 0.07 | 0.04 |

TABLE 14c
PD exposed vs. PD non-exposed

| | |
|---|---|
| SNPs | P value |

| 162 | 108 | HNF | LRT | Permutations |
|---|---|---|---|---|
| 1 | 1 | 1 | 0.03 | 0.005 |
| 0 | 1 | 1 | 0.001 | 0.001 |
| 1 | 0 | 1 | 0.1 | 0.08 |

1 - SNP included in analysis;
0 - not included

Shown are the allele frequencies and corresponding interactions between two promoter polymorphisms in PON1 (162, 108) and the promoter deletion in ACHE (HNE). Note the lower incidence of allele 1 in exposed PD patients (Table 14a) as well as its significant difference between PD patients and controls (Table 14b) and PD exposed vs. non-exposed (Table 14c).

### Example 7

Genomic Variations and Expression Differences

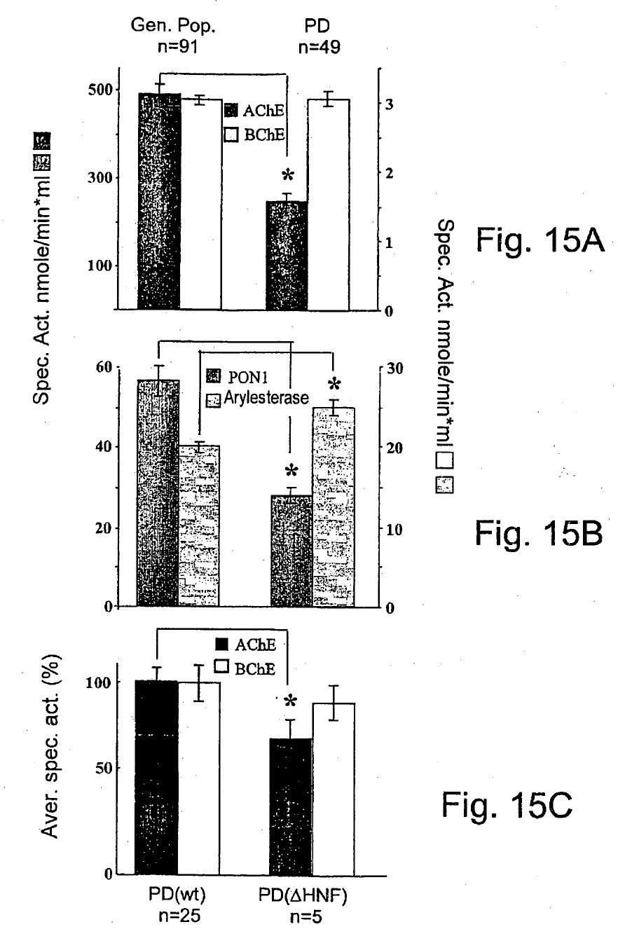

Inherited AChE overproduction was predicted to compromise the capacity of carriers to respond to anticholinesterase exposure or to stressful insults by secondary overproduction of catalytically active AChE [Shapira et al. (2000) id ibid.; Soreq and Seidman (2001) id ibid.]. Likewise, reduced PON activities [Costa et al. (2003) Annu Rev Med 54: 371-92] could be expected to subject carriers of this AChE polymorphism to additional risk because of their insufficient capacity to hydrolyze irreversible organophosphate AChE inhibitors. To evaluate the extent of susceptibility in PD patients as compared with the general population, serum cholinesterases, paraoxonase and arylesterase activities were measured in exposed and non-exposed PD patients and controls with or without the potentially predisposing polymorphisms (FIG. 15a, 15b). Both serum AChE (but not the homologous enzyme butyrylcholinesterase, BChE) and PON activity (but not protein levels reflected in arylesterase activity) were considerably lower in PD patients (P=10⁻¹⁷; =10⁻¹¹, respectively). Measuring the arylesterase activity of paraoxonase provided information on the amount of the corresponding protein; this additional test demonstrated that the over-represented haplotype in exposed PD patients directed production of normal or higher amounts but impaired activity of PON. In the rare haplotype carriers, AChE activity was greatly and significantly (P=0.012) decreased as compared with non-carrier PD patients (FIG. 15c). This in turn, supports the notion that inadequately protected AChE in the circulation of these patients increases their risk of dopaminergic. hyperactivation, leading to PD.

1. (canceled)

2. A diagnostic method of predicting susceptibility to PD, based on the detection of the “PD-susceptibility haplotype”, as herein defined, in an individual.

3. A method of screening for a genetic predisposition to PD, wherein said method involves the steps of:
(a) providing a blood sample from an individual to be screened;
(b) analyzing the DNA from the blood sample of (a) for the presence or absence of the “PD-susceptibility haplotype”, as herein defined, by appropriate means;
wherein the presence of the “PD-susceptibility haplotype” indicated a higher predisposition to PD, and the absence of the “PD-susceptibility haplotype” indicated a lower predisposition to PD, compared to a control.

4. A method of testing a blood sample of a human subject for the presence of the “PD-susceptibility haplotype”, by analyzing the DNA of said blood sample by appropriate means, wherein the presence of the “PD-susceptibility haplotype” indicated a higher predisposition of said human subject to PD, and the absence of the “PD-susceptibility haplotype” indicated a lower predisposition of said human subject to PD, compared to a control.

5. (canceled)
